(19) United States
(12) Patent Application Publication
(10) Pub. No.: US 2025/0099039 A1
(43) Pub. Date: Mar. 27, 2025

(54) VASCULAR SENSING DEVICES, SYSTEMS, AND METHODS

(71) Applicant: Veris Health Inc, New York, NY (US)

(72) Inventors: James D. Mitchell, Walnut Creek, CA (US); Lishan Aklog, Purchase, NY (US); Richard Yazbeck, Norwell, MA (US); Andrew A. Thoreson, Orono, MN (US)

(21) Appl. No.: 18/832,695

(22) PCT Filed: Jan. 26, 2023
(86) PCT No.: PCT/US23/11615

§ 371 Date: Jul. 24, 2024

Related U.S. Application Data
(60) Provisional application No. 63/303,996, filed on Jan. 28, 2022.

(51) Int. Cl. A61B 5/00; A61B 5/02; A61B 5/145; A61B 5/1455; A61B 90/00

(52) U.S. Cl. CPC A61B 5/6876; A61B 5/02028; A61B 5/14503; A61B 5/14552; A61B 5/6862; A61B 5/6882; A61B 90/06; A61B 2090/064; A61B 2560/0468; A61B 2562/0204; A61B 2562/0219; A61B 2562/0247; A61B 2562/0271

(57) ABSTRACT

Devices, systems, and methods for vascular sensing are disclosed herein. According to some embodiments, the present technology includes a method of sensing a physiological parameter. The method can include placing a device comprising an anchor and a sensor carried by the anchor in a first vascular lumen. The method further includes determining a parameter in a second vascular lumen adjacent the first vascular lumen using the sensor.

### CROSS REFERENCE TO RELATED APPLICATION

This application claims priority to U.S. Provisional Application No. 63/303,996, filed Jan. 28, 2022, and titled “VASCULAR SENSING DEVICES, SYSTEMS, AND METHODS,” which is incorporated by reference herein in its entirety.

### TECHNICAL FIELD

The present technology relates to vascular sensing devices and associated systems and methods of use.

### BACKGROUND

Prevention, detection, monitoring, and treatment of disease requires measuring one or more physiological parameters of a patient. There exists a need for improved sensing devices.

### SUMMARY

The devices and systems of the present technology may be equipped with electronic components that provide a platform for remote monitoring of one or more physiological parameters of a patient. The subject technology is illustrated, for example, according to various aspects described below, including with reference to FIGS. 1A-11B. Various examples of aspects of the subject technology are described. These aspects are provided as examples and do not limit the subject technology.

One example of the present disclosure provides a method of sensing a physiological parameter. The method includes placing an anchoring device in a first vascular lumen, the anchoring device having at least one sensor, and measuring a parameter in a second vascular lumen adjacent the first vascular lumen. In some examples, the anchoring device includes an expandable stent. In further examples, the anchoring device includes an expandable basket. In some examples, the anchoring device includes at least one metallic loop. In certain examples, the anchoring device includes a lead. In some examples, the anchoring device may be configured to apply an outward radial force to prevent migration. In further examples, the at least one sensor may include two sensors. Further, in some examples, the at least one sensor may include three sensors. In certain examples, the at least one sensor includes multiple sensors.

Another example of the present disclosure provides wherein the first vascular lumen and the second vascular lumen are separated by an interstitial space disposed therebetween. In some examples, the first vascular lumen is the superior vena cava and the second vascular lumen is the aorta. In some examples, the parameter is a first parameter and the method further includes measuring a second parameter in a third vascular lumen. In some examples, the third vascular lumen is the right pulmonary artery. In certain examples, the parameter is a first parameter and measuring the first parameter includes measuring a hemodynamic parameter from the second vascular lumen. A further example of the present disclosure provides wherein the parameter includes at least one of a pressure, a force, a rate of deflection, and an impulse. In some examples, the at least one sensor includes at least one of a temperature sensor, a pressure sensor, an accelerometer, an auditory sensor, an electrode, an oxygen saturation sensor, a photoplethysmography sensor, and an ultrasonic transducer. In some examples, the at least one sensor is configured and arranged to measure parameters in both the first and the second vascular lumens. A further example of the present disclosure includes wherein the at least one sensor is configured and arranged to directly measure a parameter from the second vascular lumen. The at least one sensor can be further configured and arranged to determine an effect of the second vascular lumen on the first vascular lumen. In further examples of the present disclosure, the at least one sensor is configured and arranged to determine the parameter in the second vascular lumen via a measurement in the first vascular lumen. Some examples of the method further include calculating at least one of a cardiac output, a flow rate in the second vascular lumen, a pressure in the second vascular lumen, an aortic pressure, and oxygen saturation. In some examples, the method can include extrapolating a respiratory parameter from the measured parameter. In certain examples, the method includes wherein the respiratory parameter includes at least one of a respiratory rate and a respiratory volume. A further example of the method includes coupling a lead to the at least one sensor, and coupling the lead to a module, the module having a processor, a memory and a battery. In some examples, the module further includes a telemetry unit capable of sending data to an external device. The module may further include at least one of an accelerometer, a temperature sensor, a pressure sensor, an auditory sensor, and an electrode.

Another example of the present disclosure provides a method of sending a physiological parameter including placing an anchoring device in a first vascular lumen, the anchoring device having at least one sensor, measuring a signal in a first vascular lumen, and analyzing the signal obtained from the first vascular lumen. The method further includes isolating a component of the signal that corresponds to a parameter in the second vascular lumen and determining a parameter in the second vascular lumen based on the component. In certain examples, the method includes calculating at least one of a cardiac output, a flow rate in the second vascular lumen, a pressure in the second vascular lumen, an aortic pressure, and oxygen saturation based on the component.

Another example of the present disclosure provides a method of sensing a physiological parameter including placing an anchoring device in a shared wall between two bodily compartments, the anchoring device having at least one sensor, and measuring a parameter via the at least one sensor. In some examples, the method includes wherein the at least one sensor includes a single sensor disposed in one of the two bodily compartments. In further examples, the method includes wherein the at least one sensor includes a single sensor disposed at the shared wall. A further example of the method includes wherein the at least one sensor includes a first sensor disposed on a first side of the shared wall, and a second sensor disposed on a second side of the shared wall, and wherein measuring a parameter includes measuring a parameter via the first and the second sensor. In some examples, the two compartments include the right atrium and the left atrium, and the shared wall is the atrial septum. In certain examples, the anchoring device includes two wide portions connected via a narrow portion, each of the two wide portions being disposed in one of two bodily compartments. In some examples, each of the two wide portions is formed of a braided nitinol wire. The at least one sensor can include one or more of at least a temperature sensor. a pressure sensor, accelerometer, an auditory sensor, an electrode, an oxygen saturation sensor, and an ultrasonic transducer. In further examples, the auditory sensor is configured, arranged and oriented to measure a sound from one or more native or artificial heart valves. In certain examples, the at least one sensor includes multiple sensors on at least one of the first side and the second side of the shared wall. In certain examples of the present disclosure, the module further includes an accelerometer disposed in the shared wall. Further, in some examples, the method includes calculating cardiac output via thermodilution.

Another example of the present disclosure provides a method of sensing a physiological parameter, the method including positioning a device in a first vascular lumen, the device including an anchor and a sensor carried by the anchor via the sensor while positioned in the first vascular lumen, and obtaining data characterizing a second vascular lumen adjacent the first vascular lumen. The method further includes, based on the data, determining a physiological parameter of the second vascular lumen. In further aspects of the method, the first vascular lumen is a vein and the second vascular lumen is an artery.

Another aspect of the invention provides a method of sensing a physiological parameter including placing an anchoring device in a shared wall between two heart chambers, the anchoring device having at least one sensor including an accelerometer, and measuring a parameter via the at least one sensor, wherein the parameter is indicative of an arrythmia.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A shows a sensing system configured in accordance with several embodiments of the present technology.

FIG. 1B shows a closer view of the sensing system shown in FIG. 1A.

FIG. 2A shows a sensing device of the sensing system of FIG. 1B positioned in a vascular lumen.

FIG. 2B is a top cross-sectional view of the sensing device and surrounding anatomy in FIG. 2A.

FIG. 3 shows a sensing system configured in accordance with several embodiments of the present technology.

FIG. 4 shows a sensing device of the sensing system of FIG. 3 positioned in a vascular lumen.

FIG. 5 shows a sensing system configured in accordance with several embodiments of the present technology. In FIG. 5, a sensing device of the sensing system is shown positioned within a patient's heart.

FIG. 6 shows a sensing system configured in accordance with several embodiments of the present technology.

FIG. 7 shows a sensing system configured in accordance with several embodiments of the present technology.

FIG. 8 shows a sensing system configured in accordance with several embodiments of the present technology.

FIG. 9 shows a sensing system configured in accordance with several embodiments of the present technology.

FIGS. 10A and 10B show a sensing system configured in accordance with several embodiments of the present technology.

FIGS. 11A and 11B show a sensing system configured in accordance with several embodiments of the present technology.

### DETAILED DESCRIPTION

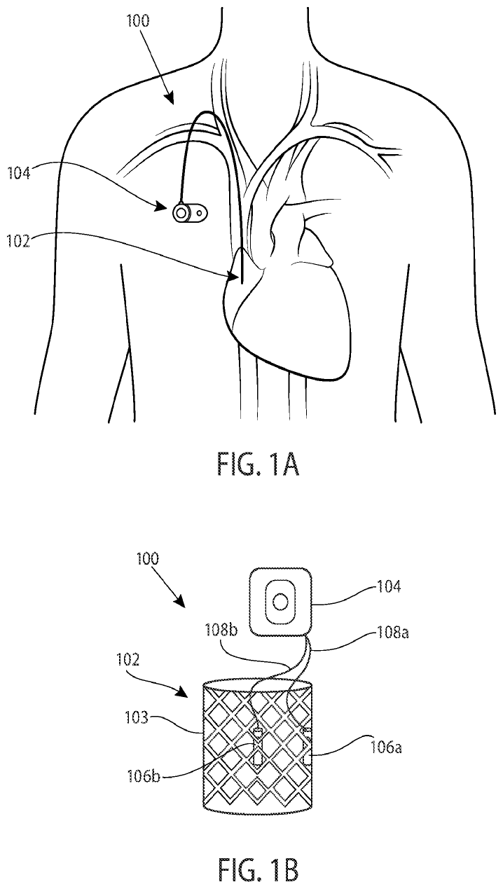

The present technology relates to vascular sensing. Some embodiments of the present technology, for example, are directed to devices, systems, and methods for measuring a physiological parameter in a first vascular lumen with a sensor positioned in a second vascular lumen adjacent the first vascular lumen. As used herein, “vascular lumen” can refer to a blood vessel (i.e., a vein or an artery) or a heart chamber (i.e., the left ventricle, the right ventricle, the left atrium, or the right atrium). Some embodiments of the present technology comprise devices, systems, and methods for measuring a physiological parameter in a first vascular lumen via a sensor positioned at or on the other side of a wall dividing the first vascular lumen and a second vascular lumen. The present technology is thus configured to measure a physiological parameter of a vascular lumen of interest with a high degree of accuracy without having to access and position a sensor (or any device) in the targeted vascular lumen. Such indirect sensing can be beneficial when trying to measure parameters in vascular lumens that are particularly difficult to access (e.g., require traversing one or more chambers of the heart) or where the presence of an interventional device presents a health risk to the patient. The present technology also includes devices, systems, and methods for directly measuring a physiological parameter from within the vascular lumen of interest. Specific details of several embodiments of the present technology are described below with reference to FIGS. 1A-11B.

FIGS. 1A and 1B show a sensing system 100 comprising a sensing device 102 and a controller 104 configured to be communicatively coupled to the sensing device 102. The controller 104 can be configured for implantation in a subcutaneous pocket (e.g., in the upper chest wall) and the sensing device 102 is configured to be positioned at a desired location within a vascular lumen. The sensing device 102 can comprise an anchor 103 configured to secure the sensing device 102 at the desired location and first and second sensors 106a. 106b (referred to collectively as “sensors 106”) carried by the anchor 103. In some embodiments, the first and second sensors 106a, 106b are coupled to the controller 104 via corresponding first and second leads 108a, 108b (referred to collectively as “leads 108”). The leads 108 can be coupled directly to the sensors 106 or indirectly via the anchor 103. In some embodiments, one or both sensors 106 are wirelessly coupled to the controller 104.

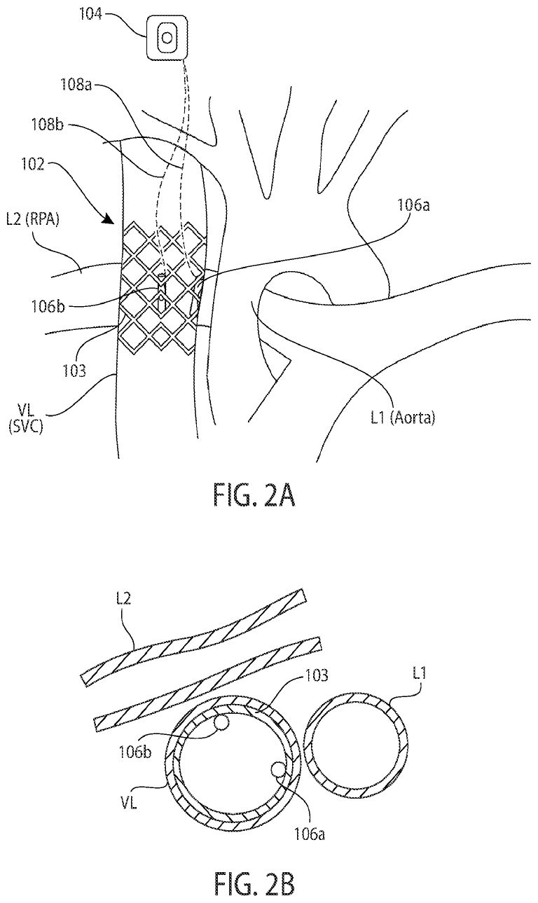

The anchor 103 can comprise an expandable structure having a low-profile state for delivery through a delivery sheath to the vascular lumen and an expanded state for anchoring at the deployment site. In the expanded state, the anchor 103 presses radially outwardly on the wall of the vascular lumen to prevent migration of the device 102. This outward radial force may also enable sensing of forces such as pressure waves/pulsations in the adjacent vessels. In some embodiments, the anchor 103 comprises a superelastic and/or resilient material (plastic or metal) that is configured to self-expand to a desired, pre-set shape when released from the delivery sheath. In these and other embodiments, the anchor 103 can be configured to expand via activation by the user, such as by use of a pull-wire, a push rod, balloon inflation, etc. As shown in FIG. 1B, in some embodiments the anchor 103 comprises a tubular, laser-cut stent including a plurality of interconnected struts that form a plurality of cells therebetween. In various embodiments, the anchor 103 comprises a braid formed of a plurality of interwoven filaments. The anchor 103 can have a variety of configurations, including a helical frame (for example as described below with reference to FIG. 2C), an expandable basket, one or more looped structures, and others. In any of the foregoing embodiments, the anchor 103 may comprise a lead.

The first and second sensors 106a, 106b can comprise the same type of sensor or may comprise different types of sensors. One, some, or all of the sensors 106 of the device 102 may comprise, for example, a temperature sensor (e.g., a thermocouple, a digital temperature sensor, a thermistor or other type of resistance temperature detector, etc.), an impedance sensor (e.g., one or more electrodes), a pressure sensor, an optical sensor, a flow sensor (e.g., a Doppler velocity sensor, an ultrasonic flow meter, etc.), an ultrasonic transducer, a photoplethysmography (PPG) sensor (e.g., pulse oximeters, etc.), a chemical sensor (e.g., an oxygen saturation sensor), a movement sensor (e.g., an accelerometer), a pH sensor, an electrocardiogram (“ECG” or “EKG”) unit, an electrochemical sensor, a hemodynamic sensor, an auditory sensor, and/or other suitable sensing device. While the device 102 shown in FIG. 1B includes two sensors 106, in other embodiments the device 102 can include more or fewer than two sensors 106 (e.g., one sensor, three sensors, four sensors, five sensors, etc.).

Based at least in part on the sensing data obtained by the sensors 106, the system 100 and/or controller 104 is configured to determine a physiological parameter of the vascular lumen in which the sensor is positioned and/or an adjacent vascular lumen. Such physiological parameters include a hemodynamic parameter, heart rate, central venous pressure, pulmonary artery pressure, aortic pressure, respiratory rate, a respiratory sound, respiratory volume, a cardiac sound, a gastrointestinal sound, speech, core temperature, an electrical signal of the heart, activity level of the patient, blood oxygenation, blood glucose, stroke volume, flow rate, cardiac output, and/or other suitable parameters. In some embodiments, the sensors 106 and/or system 100 is configured to sense one or more blood parameters, such as electrolyte concentrations, blood counts, creatinine levels, B-type natriuretic peptide (BNP) concentrations, atrial natriuretic peptide (ANP) concentrations, and others.

The controller 104 may include one or more processors, software components, and/or memory. In some examples, the one or more processors include one or more computing components configured to process measurements received from the sensors 106 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the sensors 106 to obtain one or more measurements, such as data characterizing a physiological parameter of the patient. In another example, the functions may involve processing the data to determine one or more parameters. The system 100 may be configured to continuously and/or periodically obtain measurements via the sensors 106.

The controller 104 may also include a telemetry unit configured to securely transmit data between the device 102 and one or more external computing devices. An external computing device can be a remote computing device or a local computing device, such as a mobile phone, tablet, PC, etc. In some embodiments, the controller 104 includes a localization unit configured to emit a localization signal (e.g., lights that transilluminate a patient's skin, vibration, a magnetic field, etc.) to aid a clinician in localizing the device 102 when implanted within a patient. The controller can also include a wireless charging unit (such as a coil) configured to recharge a battery (not shown) of the device 102 when in the presence of an interrogation device (e.g., a local computing device or another suitable device).

As shown in FIGS. 2A and 2B, the sensing device 102 can be configured to be positioned within a vascular lumen VL (or “primary vascular lumen VL”) such that the first and second sensors 106a, 106b are disposed at portions of the lumen wall that are in close proximity to nearby first and second vascular lumens L1 and L2 (or “secondary lumens L1 and L2”). In some embodiments the device 102 is configured to be positioned within the superior vena cava (“SVC”) such that the first sensor 106a is positioned at an inner left side of the lumen wall, adjacent the aorta (e.g., ascending aorta, aortic arch, etc.), and the second sensor 106b is positioned at an inner posterior side of the lumen wall, adjacent the right pulmonary artery (“RPA”). The walls of the first and/or second vascular lumens L1, L2 may be in contact with the wall of the primary vascular lumen VL, or may be separated from the wall of the primary vascular lumen VL by an interstitial space.

Each of the first and second sensors 106a, 106b can be configured to measure a physiological parameter of the first and second vascular lumens L1 and L2, respectively, while positioned inside of the primary vascular lumen VL. In the example shown in FIGS. 2A and 2B, each of the first and second sensors 106a, 106b is configured to measure a physiological parameter of the aorta and RPA, respectively, while positioned inside of the SVC. One or both of the first and second sensors 106a, 106b can additionally or alternatively be configured to measure a physiological parameter of the SVC.

In some embodiments, one or both of the first and second sensors 106a, 106b are configured to measure a hemodynamic parameter of a primary vascular lumen VL and/or a secondary vascular lumen. For example, the first sensor 106a can be configured to measure blood pressure within the first vascular lumen L1 and the second sensor 106b can be configured to measure blood pressure within the second vascular lumen L2. In some embodiments, the first sensor 106a is configured to measure aortic blood pressure while the second sensor 106b is configured to measure pulmonary artery pressure. In any case, the first and/or second sensors 106a, 106b can also be configured to measure pressure within the primary vascular lumen VL (such as the SVC).

To determine a physiological parameter in a secondary vascular lumen, one or both of the sensors 106 can be configured to measure deformation of the primary vascular lumen VL in response to forces on the primary vascular lumen by the secondary vascular lumen. Deformation data obtained by the sensors 106 can be communicated to the controller 104, and the controller 104 can use the deformation data to determine a physiological parameter of the secondary vascular lumen. Such a means for determining a parameter of the secondary vascular lumen can be especially useful when the primary vascular lumen and secondary vascular lumen are in contact with one another. The outer walls of the SVC and aorta, for example, may be in contact with one another such that any distension or contraction of the aorta will have a measurable effect on the SVC that can be used to determine a physiological parameter of the aorta, such as aortic blood pressure. In these and other embodiments, the first and second sensors 106a, 106b can be configured to measure and/or sense a pressure, a force, a rate of deflection, an impulse, and/or others. In those cases in which the primary and secondary vascular lumens are not in reliable contact with one another, the physiological parameter can be determined using ultrasonic sensing and/or other non-contact sensing methods.

In some embodiments, the system 100 is configured to determine at least one of a cardiac output, a flow rate in the secondary vascular lumen, a pressure in the secondary vascular lumen, an aortic pressure, and oxygen saturation. In some embodiments, the system 100 can extrapolate a respiratory parameter from the obtained measurements. In some embodiments, the respiratory parameter comprises at least one of a respiratory rate and a respiratory volume.

According to some embodiments, a method for determining a physiological parameter of a secondary vascular lumen comprises placing the device 102 in a primary vascular lumen, measuring a signal in the primary vascular lumen, analyzing the signal obtained from the primary vascular lumen, isolating a component of the signal that corresponds to a parameter in the second vascular lumen, determining a parameter in the second vessel based on the component.

In some embodiments, the device is configured to be positioned within a primary vascular lumen comprising the inferior vena cava to sense one or more parameters in a secondary vascular lumen, such as the descending aorta (or vice versa) According to several embodiments, the device is configured to be positioned within a primary vascular lumen comprising a renal vein and sense one or more parameters in a secondary vascular lumen, such as a renal artery (or vice versa). The device can be configured to be positioned within a primary vascular lumen comprising a common iliac vein and sense one or more parameters in a secondary vascular lumen, such as a common iliac artery (or vice versa). In some embodiments, the device can be configured to be positioned within a primary vascular lumen comprising a femoral vein and sense one or more parameters in a secondary vascular lumen, such as a femoral artery (or vice versa). According to several embodiments, the device is configured to be positioned within a primary vascular lumen comprising an internal jugular vein and sense one or more parameters in a secondary vascular lumen, such as a carotid artery (or vice versa). The device can be configured to be positioned within a primary vascular lumen comprising an axillary vein and sense one or more parameters in a secondary vascular lumen, such as an axillary artery (or vice versa).

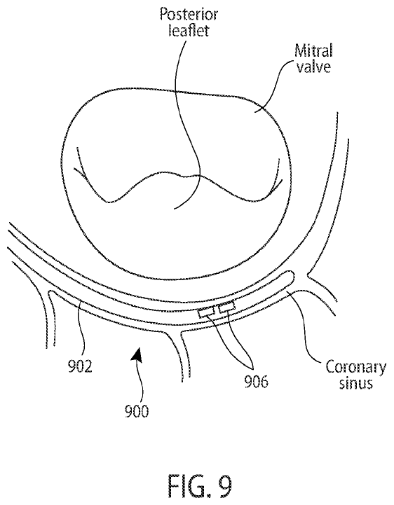

In some embodiments, the device can be configured to be positioned within a primary vascular lumen comprising the coronary sinus and sense one or more parameters in a secondary vascular lumen, such as the left heart (or vice versa). The left heart can include, for example, the left atrium, the left ventricle, the walls of the heart, and/or the mitral valve. FIG. 9, for example, shows a catheter 902 of a system 900 of the present technology positioned in the coronary sinus, adjacent the mitral valve. The catheter 900 can include one or more sensors 906. If multiple sensors are used, the different sensors can comprise the same type of sensor or may comprise different types of sensors. One, some, or all of the sensors 906 may comprise, for example, a temperature sensor (e.g., a thermocouple, a digital temperature sensor, a thermistor or other type of resistance temperature detector, etc.), an impedance sensor (e.g., one or more electrodes), a pressure sensor, an optical sensor, a flow sensor (e.g., a Doppler velocity sensor, an ultrasonic flow meter, etc.), an ultrasonic transducer, a PPG sensor (e.g., pulse oximeters, etc.), a chemical sensor (e.g., an oxygen saturation sensor), a movement sensor (e.g., an accelerometer), a pH sensor, an ECG or EKG unit, an electrochemical sensor, a hemodynamic sensor. an auditory sensor, and/or other suitable sensing device. While the catheter 902 shown in FIG. 9 includes two sensors 906, in other embodiments the catheter 902 can include more or fewer than two sensors 906 (e.g., one sensor, three sensors, four sensors, five sensors, etc.). In some embodiments, in addition to or in place of the catheter 902, the system 900 includes an anchor (not shown) configured to be temporarily or permanently positioned in the coronary sinus. The sensors 906 can be integral with and/or coupled to the anchor. The anchor can be configured primarily to provide a means for securing the sensors 906 at a desired location within the coronary sinus, or the anchoring functionality may be secondary to another interventional purpose. For example, in some embodiments the anchor is a coronary stent, an annuloplasty ring, or other interventional device configured to be positioned in the coronary sinus. In any of the foregoing embodiments, the sensor(s) 906 are configured to communicate with a controller. The controller can have the same or similar features as controller 104 and is configured to determine any of the physiological parameters disclosed herein.

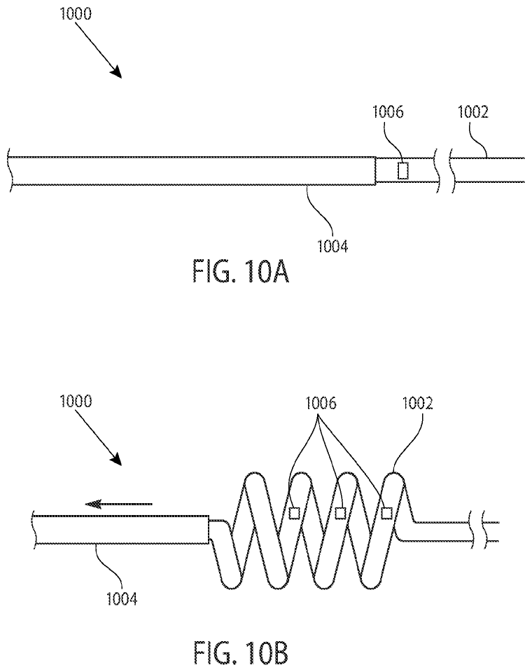

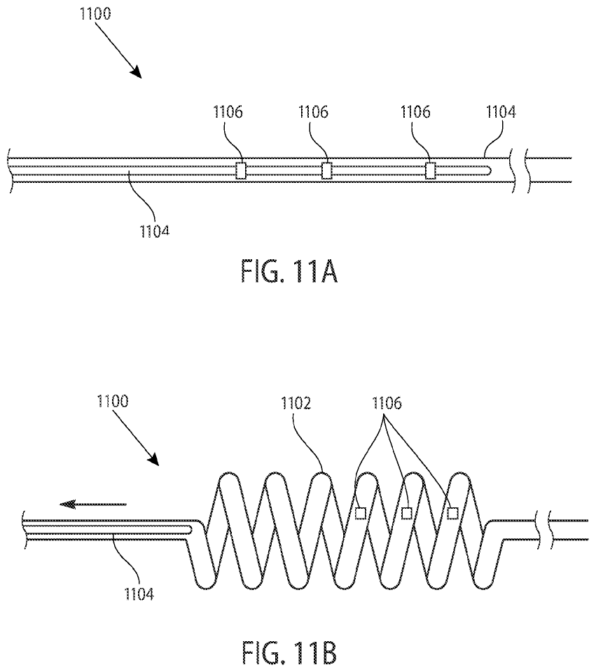

According to some embodiments of the technology. the anchor can comprise a helical structure having two or more turns and a diameter at rest that is slightly larger than the diameter of the targeted vascular lumen. As such, when deployed in the vascular lumen, the helical structure is configured to apply a radial force to the wall of the vascular lumen. Advantageously, helical structures can be advanced to the sensing site in a straightened form (e.g., via an internal obturator, external sheath, etc.) and deployed to assume a helical form (e.g., by removing the obturator, retracting the sheath, etc.). Helical structures can be repositioned relatively easily (as compared to repositioning a deployed stent) by reinserting an obturator, readvancing a sheath, etc. In some embodiments, the sensing system includes a catheter having a distal portion with one or more sensors carried thereon. The sensors can be disposed at various positions along the distal portion (and not necessarily only at the distal tip). At least the distal portion of the catheter is configured to shift between a straight configuration and a helical configuration (e.g., at least 3 turns). In the helical configuration, the turns of the distal portion of the catheter can have the same or a greater diameter than the target vessel (e.g., any of the blood vessels disclosed herein, including the SVC). The helical configuration enables the sensors to be positioned radially and/or at, near, and/or in contact with the vessel wall. The shift between straight and helical shape can be accomplished by any standard technique (shape memory/retractable sheath, retract stiffening element, etc.). For example, FIGS. 10A and 10B show a system 1000 comprising a catheter 1002 having sensors 1006 at its distal portion, where the catheter 1002 has a straight configuration until released by withdrawal of a delivery catheter 1004 that holds the distal portion in the straight configuration. FIGS. 11A and 11B show a system 1100 comprising a catheter 1102 having sensors 1106 at its distal portion, where the catheter 1102 has a straight configuration until released by withdrawal of an elongated delivery member 1104 extending through the lumen of the catheter 1102 that holds the distal portion in the straight configuration. Sensors 1006 and 1106 can be any of the sensors disclosed herein.

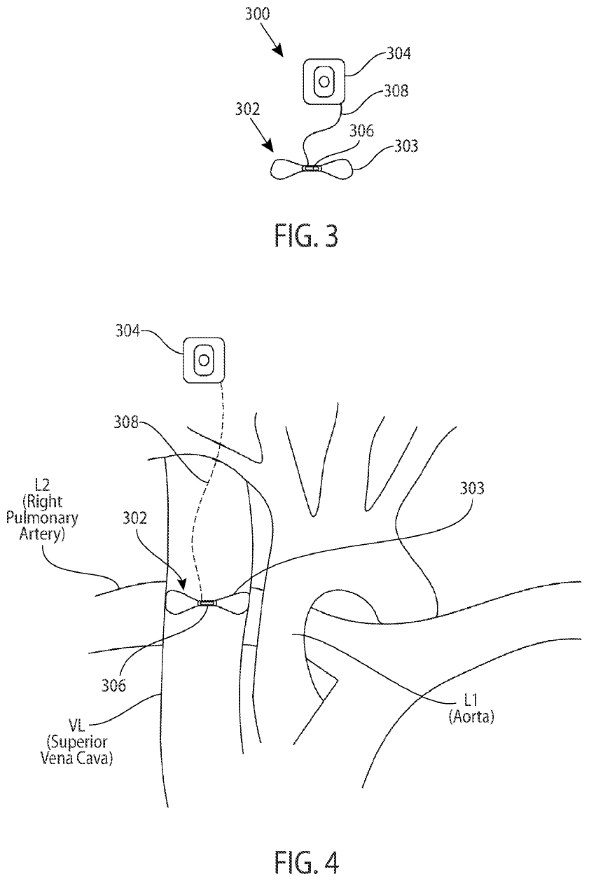

FIG. 3 shows another sensing system 300 configured in accordance with several embodiments of the present technology. The sensing system 300 comprises a sensing device 302 and a controller 304 configured to be communicatively coupled to the sensing device 302. The controller 304 can be configured for implantation in a subcutaneous pocket (e.g., in the upper chest wall) and the sensing device 302 is configured to be positioned at a desired location within a vascular lumen. The sensing device 302 can comprise an anchor 303 configured to secure the sensing device 302 at the desired location and a sensor 306 carried by the anchor 303. In some embodiments, the sensor 306 is coupled to the controller 304 via a lead 308. The lead 308 can be coupled directly to the sensor 306 or indirectly via the anchor 303. In some embodiments, the sensor 306 is wirelessly coupled to the controller 304.

According to several aspects of the present technology, for example as shown in FIG. 3, the anchor 303 can comprise two metallic loops, each extending away from opposite ends of the sensor 306. The loops can be formed of a superelastic and/or resilient material and are configured to brace the device 302 against the inner surface of the vascular lumen wall at the sensing site. In other embodiments the anchor 303 can comprise a single loop or more than two loops (e.g., three loops, four loops, etc.).

The sensor 306 can comprise an inductor coil and a pressure-sensitive capacitor that create a resonant circuit at a specific frequency. The blood pressure affects the resonant frequency, so that when the blood pressure changes, the resonant frequency changes. The controller 304 or other external device is configured to track the resonant frequency to determine the pressure in the primary vascular lumen VL.

As shown in FIG. 4, the sensing device 302 can be configured to be positioned within a vascular lumen VL (or “primary vascular lumen VL”) such that the sensor 306 is oriented with its long axis perpendicular to the longitudinal axis of the vascular lumen. In such embodiments, the anchor(s) 303 can extend radially away from the sensor 306, toward the inner surface of the vascular lumen. In some embodiments (not pictured), the sensor 306 is configured to be positioned in the vascular lumen such that its long axis is parallel with the longitudinal axis of the vascular lumen. In such embodiments, the anchor(s) 303 can extend proximally and/or distally from the sensor 306 along the longitudinal axis of the vascular lumen.

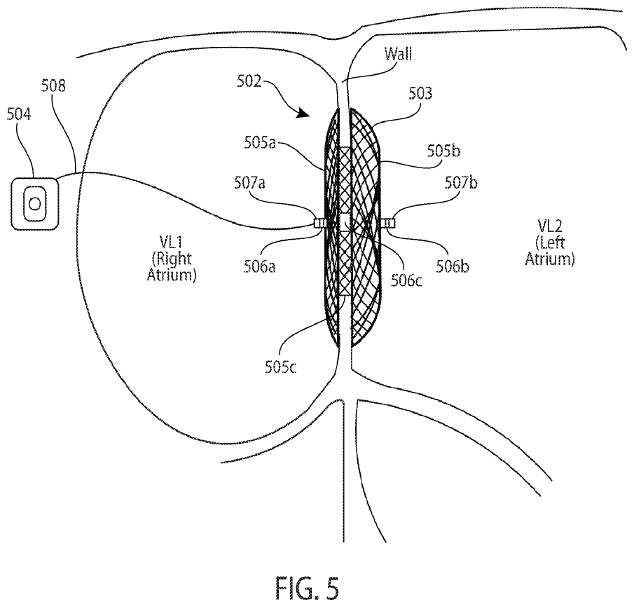

FIG. 5 shows a sensing system 500 comprising a sensing device 502 and a controller 504 configured to be communicatively coupled to the sensing device 502. The controller 504 can be configured for implantation in a subcutaneous pocket (e.g., in the upper chest wall) and the sensing device 502 is configured to be positioned at a desired location at a shared wall between two vascular lumens. The sensing device 502 can comprise an anchor 503 configured to secure the sensing device 502 at the shared wall and first, second, and third sensors 506a, 506b, 506c (referred to collectively as “sensors 506”) carried by the anchor 503. In some embodiments, the first and second sensors 506a, 506b are coupled to the controller 504 via one or more leads 508 (only one shown in FIG. 5). The leads 508 can be coupled directly to the sensors 506 or indirectly via the anchor 503. In some embodiments, one, some, or all of the sensors 506 are wirelessly coupled to the controller 504.

The anchor 503 can comprise an expandable structure having a low-profile state for delivery through a delivery sheath to the shared wall and an expanded state for anchoring within an opening in the shared wall. In some embodiments, the anchor 503 comprises a superelastic and/or resilient material (plastic or metal) that is configured to self-expand to a desired, pre-set shape when released from the delivery sheath. In these and other embodiments, the anchor 503 can be configured to expand via activation by the user, such as by use of a pull-wire, a push rod, etc.

In some embodiments, for example as shown in FIG. 5, the anchor 503 comprises a mesh formed of a tubular braid that has been shape set to assume a predetermined shape when released from the constraints of a delivery sheath. The proximal and distal ends of the braid are held together by proximal and distal hubs 507a and 507b, respectively. The mesh can have a first broad portion 505a configured to be positioned on one side of the wall, a second broad portion 505b configured to be positioned on the other side of the wall, and a narrow portion 505c extending between the first and second broad portions 505a, 505b and configured to be positioned within the opening in the wall. Each of the first and second broad portions 505a, 505b can have a cross-sectional dimension that is greater than a cross-sectional dimension of the opening in the wall. As such, positioning the first and second broad portions 505a, 505b on either side of the wall prevents the device 502 from dislodging through the opening.

The sensors 506 can be disposed on one, some, or all of: the proximal hub 507a, the distal hub 507b, the first broad portion 505a, the second broad portion 505b, and the narrow portion 505c. As shown in FIG. 5, in some embodiments the first sensor 506a is disposed at the proximal hub 507a, the second sensor 506b is disposed at the distal hub 507b, and the third sensor 506c is disposed at the narrow portion 505c. As such, when the device 502 is secured at the wall, the first sensor 506a is positioned in the first vascular lumen VL1 (on one side of the wall), the second sensor 506b is positioned in the second vascular lumen VL2 (on the other side of the wall), and the third sensor 506c is positioned partially or completely within the opening extending through the wall. According to some methods of use, the first vascular lumen VL1 can be the right atrium, the second vascular lumen VL2 can be the left atrium, and the wall can be the atrial septum. The device 502, for example, can be configured to be positioned across a patent foramen ovale (“PFO”) to stop the abnormal flow of blood between the two atrial chambers of the heart.

The first, second, and third sensors 506a, 506b, 506c can comprise the same sensor or may comprise different types of sensors. One. some, or all of the sensors 506 of the device 502 may comprise, for example, a temperature sensor (e.g., a thermocouple, a digital temperature sensor, a thermistor or other type of resistance temperature detector, etc.), an impedance sensor (e.g., one or more electrodes), a pressure sensor, an optical sensor, a flow sensor (e.g., a Doppler velocity sensor, an ultrasonic flow meter, etc.), an ultrasonic transducer, a photoplethysmography (PPG) sensor (e.g., pulse oximeters, etc.), a chemical sensor (e.g., an oxygen saturation sensor), a movement sensor (e.g., an accelerometer), a pH sensor, an electrocardiogram (“ECG” or “EKG”) unit, an electrochemical sensor, a hemodynamic sensor, an auditory sensor, and/or other suitable sensing device. Although the device 502 is shown in FIG. 5 having three sensors 506, the device 502 may have more or fewer sensors. For example, in some embodiments the device 502 includes a single sensor positioned at one side of the wall or a single sensor disposed at the narrow portion 505c. In certain embodiments, the device 502 includes only two sensors, four sensors, five sensors, six sensors, etc. In these and other embodiments, the device 502 can comprise multiple sensors on each of the first and second broad portions 505a, 505b such that, when implanted, the device 502 comprises multiple sensors on each side of the shared wall.

Based at least in part on the sensing data obtained by the sensors 506, the system 500 and/or controller 504 is configured to determine a physiological parameter of one or both vascular lumens, such as a hemodynamic parameter, heart rate, central venous pressure, pulmonary artery pressure, aortic pressure, respiratory rate, a respiratory sound, respiratory volume, a cardiac sound, a gastrointestinal sound, speech, core temperature, an electrical signal of the heart, activity level of the patient, blood oxygenation, blood glucose, stroke volume, flow rate, cardiac output, and/or other suitable parameters.

Similar to controllers 104 and 304, the controller 504 may include one or more processors, software components, and/or memory (not shown). In some examples, the one or more processors include one or more computing components configured to process measurements received from the sensors 506 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the sensors 106 to obtain one or more measurements, such as data characterizing a physiological parameter of the patient. In another example, the functions may involve processing the data to determine one or more parameters. The system 500 may be configured to continuously and/or periodically obtain measurements via the sensors 506.

The controller 504 may also include a telemetry unit configured to securely transmit data between the device 502 and one or more external computing devices. An external computing device can be a remote computing device or a local computing device, such as a mobile phone, tablet, PC, etc. In some embodiments, the controller 504 includes a localization unit configured to emit a localization signal (e.g., lights that transilluminate a patient's skin, vibration, a magnetic field, etc.) to aid a clinician in localizing the device 502 when implanted within a patient. The controller can also include a wireless charging unit (such as a coil) configured to recharge a battery (not shown) of the device 502 when in the presence of an interrogation device (e.g., a local computing device or another suitable device).

In some embodiments, system comprises one or more auditory sensors configured to be positioned at or in the heart. For example, in some embodiments the system includes a first auditory sensor positioned at one location at and/or within the heart and a second auditory sensor positioned at and/or within the heart. In some embodiments, the system can be any of systems 500, 600, and 700. More than two sensors can be used. For example, the system can be configured to analyze sounds at or near the tricuspid and/or mitral valves for signs of regurgitation or flow across a narrowed valve. For example, in some embodiments, the system comprises an auditory sensor arranged and oriented to measure a sound from one or more native or artificial heart valves. The auditory sensor(s) and/or associated controller can be configured to detect the sound of a “leaky valve” with blood regurgitation (commonly heard as a murmur with a stethoscope) which can indicate abnormal flow across valves. Auditory sensors can beneficially provide insight into function of valves from both sides of the heart. In the right and left atria, the sensors can be positioned adjacent to the tricuspid and mitral valves, respectively. Valves have two functions: allowing the passage of blood in the open configuration and stopping the back flow (regurgitation) of blood in the closed position. A problem in one of these two functions will lead to abnormal heart sounds (murmur). A narrow valve causes turbulent flow in the open position leading to a “flow murmur.” A leaky valve will great jets of regurgitation with characteristic sounds.

In some embodiments, system comprises one or more pressure sensors configured to be positioned at or in the heart. For example, in some embodiments the system includes a first pressure sensor positioned at one location at and/or within the heart and a second pressure sensor positioned at and/or within the heart. In some embodiments, the system can be any of systems 500, 600, and 700. More than two sensors can be used. Such embodiments can be beneficial when treating patients with congestive heart failure (CHF) that are at risk for both venous congestions and arrhythmia. By sensing pressure in the right atrium and the left atrium, for example. the present technology is configured to directly measure the amount of venous congestion which worsens with worsening CHF. In some embodiments, the system is configured to compare a function parameter (e.g., indicative of the heart's ability to eject blood into the circulation) of the right and left heart independently. The function parameter could be, for example, cardiac output or ejection fraction. If these parameters decrease, fluid backs up (congestion), which can lead to congestive heart failure.

In some embodiments, the first, second, and/or third sensor 506a, 506b, 506c comprises an electrode. The electrode can be configured to be positioned in direct contact with the cardiac tissue. The electrode(s) can be configured to sense data that enables the system and/or controller to provide a detailed cardiac rhythm analysis as it would be in direct contact with the cardiac tissue. For example, the electrodes can enable detection of abnormal cardiac rhythms.

According to various embodiments, the third sensor 506c comprises an accelerometer disposed in the shared wall. The accelerometer(s) can be configured to provide information about cardiac contractility, which diminishes with worsening heart failure. Additionally, the accelerometer(s) can be configured to pick up characteristic cardiac wall motion associated with certain arrhythmias such as atrial fibrillation. Some methods of use include placing an anchoring device in a shared wall between two heart chambers where the anchoring device has at least one sensor comprising an accelerometer. The method further includes measuring a parameter via the at least one sensor, where the parameter is indicative of an arrythmia.

In some embodiments, the present technology can be configured to calculate cardiac output via thermodilution. For example, several systems of the present technology include a first sensor configured to be positioned at and/or in the right side of the heart (e.g., in and/or on the right atrium, right ventricle, and/or associated valves) and a second sensor configured to be positioned at and/or in a left side of the hear (e.g., in and/or on the left atrium, left ventricle, and/or associated valves). Such systems may comprise, for example, any of the anchors 503, 603, or 703 of FIGS. 5-7 and the first and second sensors could be, for example, the first and second sensors 506a, 506b. 606a, 606b, 706a, and 706b of any of FIG. 5, 6, or 7. It will be appreciated that systems for measuring cardiac output via thermodilution in accordance with the present technology can have configurations and components different than those shown and described with respect to FIGS. 5, 6, and 7. According to some embodiments, the first and second sensors comprise temperature sensors. In such embodiments, a fluid (e.g., warm saline) can be injected into the circulation at one side of the heart (the right side or the left side), and the temperature of the circulation at that side of the heart and the temperature of the circulation at the other side of the heart can be measured via the first and second sensors. The system may also measure a baseline temperature (pre-injection of fluid) of the circulation at one or both sides of the heart via the sensors. The system and/or controller can be configured to determine cardiac output based on the absolute temperatures, change in temperatures, and/or comparison in absolute and/or change in temperatures of the two sides.

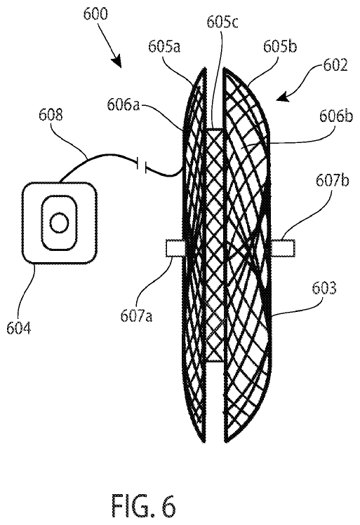

FIG. 6 shows a sensing system 600 similar to the system 500 shown and described with reference to FIG. 5 except that in system 600, the first and second sensors 606a, 606b of the device 602 are disposed on the first and second broad portions 605a, 605b, respectively, of the mesh, rather than on the proximal and distal hubs 607a, 607b. The device 602 of FIG. 6 also does not include a third sensor at the narrow portion 605c.

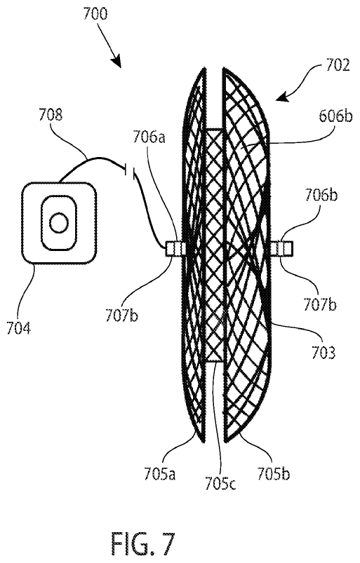

FIG. 7 shows a sensing system 700 similar to system 500 except that the device 702 of FIG. 7 does not include a third sensor at the narrow portion 705c. The configuration shown in FIG. 7 could be used to calculate cardiac output via thermodilution, among other uses. In such embodiments, the first sensor 706a would comprise the left heart sensor and the second sensor 706b would comprise the right heart sensor. It will be appreciated that other configurations of sensors and anchors could be used to measure cardiac output via thermodilution.

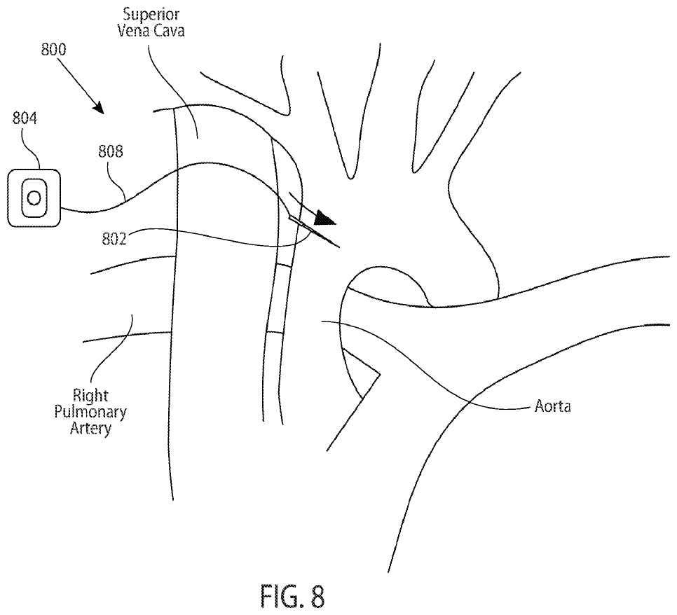

FIG. 8 shows another sensing system 800 configured in accordance with several embodiments of the present technology. The sensing system 800 comprises a sensing device 802 and a controller 804 configured to be communicatively coupled to the sensing device 802 (e.g., wirelessly or via a lead 808). The controller 804 can be configured for implantation in a subcutaneous pocket (e.g., in the upper chest wall) and the sensing device 802 is configured to pierce a wall of a vascular lumen that has been targeted for sensing. The sensing device 802, for example, can have a sharpened end and can be configured to penetrate through a wall of a vascular lumen, such as the aorta.

### CONCLUSION

Although many of the embodiments are described above with respect to devices, systems, and methods for measuring one or more physiological parameters of a patient via a sensor positioned in a vascular lumen, the technology is applicable to other applications and/or other approaches. For example, the present technology includes devices configured to be positioned in bodily lumens that are not part of the vasculature and/or heart (or “non-vascular lumens”) and configured to measure physiological parameters in adjacent vascular or non-vascular lumens. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-11B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about.” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method of sensing a physiological parameter, comprising:
placing an anchoring device in a first vascular lumen, the anchoring device having at least one sensor; and
measuring a parameter in a second vascular lumen adjacent the first vascular lumen.

2. The method of claim 1, wherein the anchoring device comprises an expandable stent.

3. The method of claim 1, wherein the anchoring device comprises an expandable basket.

4. The method of claim 1, wherein the anchoring device comprises at least one metallic loop.

5. The method of claim 1, wherein the anchoring device comprises a lead.

6. The method of claim 1, wherein the anchoring device is configured to apply an outward radial force to prevent migration.

7. The method of claim 1, wherein the at least one sensor comprises two sensors.

8. The method of claim 1, wherein the at least one sensor comprises three sensors.

9. The method of claim 1, wherein the at least one sensor comprises multiple sensors.

10. The method of claim 1, wherein the first vascular lumen and the second vascular lumen are separated by an interstitial space disposed therebetween.

11. The method of claim 1, wherein the first vascular lumen is the superior vena cava and the second vascular lumen is the aorta.

12. The method of claim 1, wherein the parameter is a first parameter and the method further comprises measuring a second parameter in a third vascular lumen.

13. The method of claim 12, wherein the third vascular lumen is the right pulmonary artery.

14. The method of claim 1, wherein the parameter is a first parameter and measuring the first parameter comprises measuring a hemodynamic parameter from the second vascular lumen.

15. The method of claim 1, wherein the parameter comprises at least one of a pressure, a force, a rate of deflection, and an impulse.

16. The method of claim 1, wherein the at least one sensor includes at least one of a temperature sensor, a pressure sensor, an accelerometer, an auditory sensor, an electrode, an oxygen saturation sensor, a photoplethysmography sensor, and an ultrasonic transducer.

17. The method of claim 1, wherein the at least one sensor is configured and arranged to measure parameters in both the first and the second vascular lumens.

18. The method of claim 1, wherein the at least one sensor is configured and arranged to directly measure a parameter from the second vascular lumen.

19. The method of claim 1, wherein the at least one sensor is configured and arranged to determine an effect of the second vascular lumen on the first vascular lumen.

20. The method of claim 1, wherein the at least one sensor is configured and arranged to determine the parameter in the second vascular lumen via a measurement in the first vascular lumen.
